(19) United States
(12) Patent Application Publication
(10) Pub. No.: US 2021/0370066 A1
(43) Pub. Date: Dec. 2, 2021

(54) NEUROMODULATION AND/OR NEUROSTIMULATION SYSTEM

(71) Applicant: ONWARD MEDICAL B.V., Eindhoven (NL)

(72) Inventors: Miroslav CABAN, Renens (CH); Joachim von ZITZEWITZ, Bussigny (CH)

(73) Assignee: ONWARD MEDICAL B.V., Eindoven (NL)

(21) Appl. No.: 17/303,245

(22) Filed: May 25, 2021

(30) Foreign Application Priority Data
May 26, 2020 (EP) 20020250.5

(51) Int. Cl. A61N 1/36

(52) U.S. Cl. CPC A61N 1/36062; A61N 1/36031; A61N 1/36135; A61N 1/36002; A61N 1/36003; A61N 1/36139; A61N 1/36114

(57) ABSTRACT

A system for neuromodulation or neurostimulation comprising at least one sensing unit configured to provide a sensor signal correlating with a physiological value describing neurological function or dysfunction of a patient, at least one control unit, at least one stimulation unit, and at least one of at least one Central Nervous System stimulation module for providing Central Nervous System stimulation or at least one Peripheral Nervous System stimulation module for providing Peripheral Nervous System stimulation, wherein the control unit is configured to detect the neurological dysfunction based on the sensor signal and to trigger the neuromodulation or neurostimulation. The disclosure further relates to a method for providing neuromodulation or neurostimulation and the use of a neuromodulation system in the method for the treatment of a patient.

### CROSS-REFERENCE TO RELATED APPLICATION

This application claims the benefit of priority of European Patent Application No. EP20020250.5, filed May 26, 2020, titled “NEUROMODULATION AND/OR NEUROSTIMULATION SYSTEM,” the entire disclosure of which is incorporated herein by reference.

### BACKGROUND

The spinal cord is an integral part of the central nervous system (CNS). Spinal cord injury (SCI), and other disorders or injuries (e.g. stroke, Parkinson Disease, brain injury, cerebral palsy, multiple sclerosis, autonomic failure, autonomic neuropathy or cancer of the neurological tissue) often results in motor deficits. For instance, SCI interrupts the communication between the spinal cord and supraspinal centers, depriving these sensorimotor circuits from the excitatory and modulatory drives necessary to produce movement.

Normal autonomic function is critically dependent on the interaction between supraspinal centers and spinal autonomic components. Descending neuronal projections from the brainstem and hypothalamus regulate sympathetic and parasympathetic activities at the spinal cord level. Traumatic SCI or disorders such as stroke, Parkinson Disease, multiple sclerosis, autonomic failure, autonomic neuropathy or cancer of the neurological tissue can interrupt connections between higher centers and the spinal cord, resulting not only in somato-motor and sensory deficits but also in autonomic dysfunctions. In particular, SCI or the above-mentioned disorders often result in disconnection of some, most, or all descending pathways that carry signals responsible for regulating arterial blood pressure, heart rate, respiratory function, sexual function, thermoregulation, gut function, bladder function, bowel function, production of body fluids (saliva, sweat, tears), balance of water and electrolytes (sodium and calcium), metabolism, or digestion.

From a historical perspective, autonomic dysfunctions have not been the focus of basic or clinical research when compared to the amount of attention that “curing paralysis” has received. However, in recent decades autonomic disorders after SCI have drawn more investigations as researchers and clinicians began to pronounce their clinical priority. Autonomic dysfunction following SCI is a potentially life-threatening condition that leads to blood pressure instability and ensuing chronic dysfunction of the heart and vasculature. Most SCI patients experience multiple drastic, blood pressure fluctuations daily, and rank this as a top healthcare priority.

Neuromodulation, including neurostimulation by means of neuromodulation system/neurostimulation system is a well-established neuromodulatory/neurostimulatory therapy not only for restoring locomotion/motoric function after spinal cord injury or central nervous system diseases, but also for treating inter alfa pain or restoring autonomic function.

Known stimulation systems for treating patients after spinal cord injury (e.g. to restore motor function or blood pressure) mainly use either CNS stimulation, including Epidural Electrical Stimulation (EES), or Peripheral Nerve System (PNS) Stimulation, including Functional Electrical Stimulation (FES).

EES is known to restore motor control in animal and human models and has more particularly been shown to restore locomotion after spinal cord injury by artificially activating the neural networks responsible for locomotion below the spinal cord lesion. (Capogrosso et al., A Computational Model for Epidural Electrical Stimulation of Spinal Sensorimotor Circuits, Journal of Neuroscience 4 Dec. 2013, 33 (49) 19326-19340, Courtine et al., Transformation of nonfunctional spinal circuits into functional states after the loss of brain input, Nat Neurosci. 2009 Oct.; 12(10): 1333-1342. Maraud et al, Mechanisms Underlying the Neuromodulation of Spinal Circuits for Correcting Gait and Balance Deficits after Spinal Cord Injury, Neuron Volume 89, Issue 4, 814-828, 17 Feb. 2016). EES does not directly stimulate motor-neurons but the afferent sensory neurons prior to entering into the spinal cord. In this way, the spinal networks responsible for locomotion are recruited indirectly via those afferents, restoring globally the locomotion movement by activating the required muscle synergies. The produced movement is functional; however, due to relatively poor selectivity (network activation instead of selective targeting of key muscles) the controllability is low, and the imprecisions hinder fluidity and full functionality in the potential space of the movement. EES may also be applied for restoring autonomic function after SCI.

PNS stimulation systems used to date in the clinic are known as FES that provides electrical stimulation to target muscles with surface electrodes, either directly through stimulation of their motor fibers (neuro-muscular stimulation), or through a limited set reflexes (practically limited to the withdrawal reflex) or by transcutaneously stimulating the peripheral nerves. The resulting muscle fatigue has rendered FES unsuitable for use in daily life. Furthermore, successes have remained limited through cumbersome setups when using surface muscle stimulation, unmet needs in terms of selectivity (when using transcutaneous nerve stimulation) and a lack of stability (impossible to reproduce exact electrode placement on a daily basis when stimulating muscles, moving electrodes due to clothes, sweating).

A system can deliver adaptive electrical spinal cord stimulation (in particular EES) to facilitate and restore locomotion after neuromotor impairment. Inter alia, a closed-loop system for real-time control of EES may comprise means for applying to a subject neuromodulation with adjustable stimulation parameters, means being operatively connected with a real-time monitoring component comprising sensors continuously acquiring feedback signals from subject, signals providing features of motion of a subject, system being operatively connected with a signal processing device receiving feedback signals and operating real-time automatic control algorithms, signal processing device being operatively connected with means and providing means with new stimulation parameters, with minimum delay. Known systems can improve consistency of walking in a subject with a neuromotor impairment.

Known devices and algorithms are used for controlling an autonomic function in an individual by providing CNS stimulation. For example, in such a device, a controller device can utilize physiological measurements (such as blood pressure) to regulate spinal cord electrical stimulation to stabilize blood pressure. A control interface and algorithm for controlling an autonomic function in a subject. For example, an algorithm can utilize physiological measurements (such as blood pressure) to regulate spinal cord electrical stimulation to stabilize blood pressure. The neuronal structures involved may be located within the T1 to S5 segments of the spinal cord, for example. Stimulation may be configured to control a particular function by selecting electrodes or the nature of the stimulation.

Also known is an electric stimulation apparatus for treating hypotension of patients with spinal cord injury and a method for treating hypotension. An electric stimulation apparatus comprises: a blood pressure measuring means for continuously measuring a blood pressure of a. subject; an electric current application means comprising an electrode for intermittently applying an electric current to skin of the subject; and a control means for controlling the electric current application means so as to maintain the blood pressure at a predetermined target blood pressure value by activating the electric current application means when the subject blood pressure is equal to or less than the target blood pressure value. The electrode can be positioned within inguinal, femoral, lumbar and lower abdominal regions of the subject.

It is an object of the present disclosure to provide an improved system and method to control impaired neurological function, including impaired autonomic function, by limiting side effects when applying neuromodulation to a patient suffering from autonomic dysfunction.

This object is solved according to the present disclosure with a system for neuromodulation or neurostimulation. Accordingly, a system for neuromodulation or neurostimulation comprising at least one sensing unit configured to provide a sensor signal correlating with a physiological value describing neurological function or dysfunction of a patient, at least one control unit, at least one stimulation unit, and at least one of at least one CNS stimulation module for providing CNS stimulation or at least one PNS stimulation module for providing PNS stimulation, wherein the control unit is configured to detect the neurological dysfunction based on the sensor signal and to trigger the neuromodulation or neurostimulation.

### DESCRIPTION

The present disclosure relates to a system for neuromodulation, including neurostimulation, for the treatment of a subject, in the field of improving, for example, after disorders or injuries of the central nervous system (CNS), such as the spinal cord.

The present disclosure further relates to a method for providing neuromodulation or neurostimulation and the use of a system for neuromodulation or neurostimulation in a method for the treatment of a patient after disorders or injuries of CNS, such as SCI.

The disclosure is based on the basic idea that a neurostimulation system is provided which specifically targets and modulates neurons, smooth muscles, or muscles responsible for or involved in autonomous control in a way that the system enables precise and optimized control over autonomous control after SCI. The use of a hardware system comprising at least one sensing unit, control unit, stimulation unit and CNS stimulation module or a PNS stimulation module enables optimized neurostimulation by sensing physiological parameters of neurological function, including autonomic function or dysfunction of a patient, defining or using a target value of neurological function, including autonomic function and identification of optimal stimulation parameters for restoring neurological function, including autonomous function to minimize the difference between the sensed physiological parameters of neurological function, including autonomic function or dysfunction and the respective target value. A central role may play the control unit, comparing the physiological status of the patient with the respective target value, and calculating optimal stimulation parameters, such as electrode configuration, frequency, amplitude or pulse width, for equaling the physiological parameters to the target value, and at the same time minimizing effects on other functions or responses to the stimulation, e.g. muscle function. A further central role may play the stimulation unit, providing neuromodulation via the CNS stimulation module or PNS stimulation module, in order to provide optimal stimulation or minimize side effects of stimulation. Therefore, it is possible to improve a neuromodulation system, e.g. in the field of improving recovery after neurological disorders like SCI, especially in that neuromodulation or neurostimulation can be provided in almost any environment and in daily life, adapted to the patient's needs and providing the needed assistance in training and daily life for the patient, also adjusted to the progress of the rehabilitation of the patient.

One further advantage of the system according to the present disclosure is that the system could be combined, connected or included in a system for restoring motoric function after spinal cord injury or disorders (e.g. stroke, Parkinson Disease, cerebral palsy, brain injury, multiple sclerosis, autonomic failure, autonomic neuropathy, or cancer of the neurological tissue which impair operation of descending pathways that normally facilitate control of motoric function).

Autonomic function may be blood pressure (systolic blood pressure, diastolic blood pressure, mean arterial blood pressure), heart rate, respiratory function, sexual function, thermoregulation, gut function, bladder function, bowel function, production of body fluids (saliva, sweat, tears), balance of water and electrolytes (sodium and calcium), metabolism, or digestion.

Without further specification ‘blood pressure’ often may refer to the pressure in arteries of the systemic circulation. Blood pressure may usually be expressed in terms of at least one of systolic blood pressure, over diastolic blood pressure, or over mean arterial blood pressure, i.e. an average blood pressure in an individual during a single cardiac cycle and may be measured in millimeters of mercury (mmHg, above the surrounding atmospheric pressure).

Blood pressure monitoring may comprise monitoring a parameter value such as diastolic blood pressure, systolic blood pressure, diastolic blood pressure and a systolic blood pressure, mean arterial pressure, or the like.

Dangerous decreases (i.e. hypotension) or elevations (i.e. hypertension) of blood pressure, including arterial blood pressure, may occur as a consequence of SCI, as the spinal cord neurons responsible for blood pressure control no longer have the capacity to maintain blood pressure at a normal physiological level. Hypotension may, for instance, lead to dizziness, disorientation, reduction in cognitive functioning, loss of consciousness, a predisposition to strokes, or heart attacks. Hypertension may, for instance, lead to heart attacks, strokes, or sub-clinical vascular consequences.

Individuals with severe SCI at the level of T6 (spinal segment) or above, or at the level of T10 or above (spinal segment), often suffer from autonomic dysfunction or dysregulated cardiovascular control, or both. Usually, the higher the level of the spinal cord injury the more severe is the hypertension in autonomic dysfunction.

Consistent with disclosed embodiments, neurological functions or dysfunctions that may be treated or stabilized with the neuromodulation system may be spasticity, clonus, pain, or any combination of them.

Consistent with disclosed embodiments, the system may be used to continuously control or maintain neurological function, including autonomic function of a patient. In some embodiments, the system may be used to prevent critical situations, such as attacks of autonomic dysreflexia.

Consistent with disclosed embodiments, the sensing unit may comprise at least one sensor. In some embodiments, the sensing unit may be at least one of a blood pressure sensor, a pulse oximeter, a heart-rate sensor, a breathing rate sensor, a body temperature sensor, a pressure sensor (for instance for sensing bladder internal pressure), a pH sensor, a barometer, an inertial measurement unit, an accelerometer, a gyroscope, a chemical concentration sensor (glucose, water, sodium, calcium), a hormone sensor, a metabolites sensor, a sweat sensor, a gastrointestinal movement sensor, a hydration sensor, an electrolyte sensor, an arterial stiffness sensor, a urine concentration sensor, a urine volume sensor, a bladder volume sensor, a conductivity sensor, a capacitance sensor, a strain gauge sensor, or a genital blood flow sensor. Such a sensing unit may enable that the system is a closed-loop system, where feedback mechanisms are based on sensed data.

Consistent with disclosed embodiments, at least two sensors may from a sensor network. Thus, as a non-limiting example, at least one of a blood pressure sensor, a pulse oximeter, a heart-rate sensor, a body temperature sensor may be combined for sensing patient data. This may enable an optimal monitoring of more than one physiological status or performance, based on which an optimal therapy may be provided by the system.

Consistent with disclosed embodiments, at least one of the components of the system for neuromodulation or neurostimulation may be implantable. The at least one sensor unit or sensor may be invasive or non-invasive. In other words, the at least one sensor may be at least partially implantable or implanted. Alternatively, the at least one sensor may be not implanted or not implantable. In some embodiments, the at least one sensor is a digital or analog sensor system. A sensor network may generally comprise both at least one at least partially implanted or implantable sensor and at least one non-implantable or non-implanted sensor.

A blood pressure sensor may measure or monitor systolic, diastolic, or mean arterial pressure (or also spinal cord perfusion pressure) of the patient.

Consistent with disclosed embodiments, an implanted or implantable sensor, a non-implantable or non-implanted sensor may be, but is not limited to, an upper arm blood pressure monitor system, a wrist blood pressure monitor system, or a finger blood pressure monitor system. The sensor may measure or monitor a blood pressure signal indicative for a blood pressure measurement. In general, the at least one sensor may provide continuous monitoring of blood pressure, sporadic monitoring of blood pressure, or measuring or monitoring blood pressure in preset time intervals.

Consistent with disclosed embodiments, the sensor may comprise at least one of an electrocardiogram (ECG), an electroencephalogram (EEG), an electromyogram (EMG), a patch clamp, a voltage clamp, an extracellular single-unit recorder, or a recorder of local field potentials. An ECG may sense the cardiac rhythm (heart rate) of a patient. The cardiac rhythm may then be related to a status of cardiac output, e.g. a status with good cardiac output, a status of medium cardiac output, or a status of bad cardiac output. The ECG may comprise sensors placed on the skin of the patient, which enables immediate, non-invasive and low-cost sensing of physiological parameters (cardiac rhythm, heart rate) of the patient.

An EEG may sense summed electrical activity of the brain by recording the voltage fluctuations on the head surface. The EEG may comprise sensors placed on the skin/head of the patient, which also enables immediate, non-invasive and low-cost sensing of physiological parameters (electrical brain activity) of the patient. Sensing patient physiological parameters by electroencephalography may enable to correct noisy ECG or other signals (obtained by blood pressure sensing, etc.). Physiological parameters sensed by the sensing unit, e.g. blood pressure or cardiac rhythm (by ECG) may be not reliable due to unexpected noise that distort the signals. It may be possible that the system, including the control unit, corrects such artifacts, e.g. ECG artifacts, by using the EEG signals.

Further, the sensing unit is configured and arranged for, or the control unit is configured and arranged for optimizing stimulation parameters for stabilization or treatment of the neurological function, including autonomic function, of the patient. In some embodiments, stimulation parameters may comprise electrode configuration, frequency, amplitude, or pulse with of at least one pulse train. Thus, the sensing unit that can directly or indirectly measure the effect of the provided stimulation on the desired neurological system, including autonomic system, or the control unit that can perform an approximate mapping may enable automatic or semi-automatic optimization of the stimulation parameters.

Further, the sensing unit may comprise at least one trigger, e.g. a manual trigger, to be triggered by the patient and providing a signal describing dysfunction of the autonomic function of the patient. The trigger may, as a non-limiting example, comprise an emergency button. For instance, when a patient recognizes feelings or conditions including but not limited to dizziness, disorientation, nausea, reduction in cognitive functioning, beginning loss of consciousness, sweating, shortness of breath, blue lips, no sphincter relaxation, loss of urine, loss of feces, or spasticity, triggering the trigger may enable to restore autonomic function or at least partially remedy the above-mentioned symptoms. This may be advantageous when the system is used as an open-loop system without adaptation of stimulation parameters to feedback data or when the control mechanisms of the system, when used as a closed-loop system, fail.

The stimulation unit may comprise a pulse generator, e.g. an implantable pulse generator, or one electrode or a plurality of electrodes. Subsets of electrodes may be defined as any value x out of the range 0 to n, n being the number of electrodes available. The plurality of electrodes may be arranged in an array, e.g. in an electrode array on a stimulation lead/paddle. Such a stimulation lead may be a lead paddle or a lead wire or any kind of lead or carrier(s) having at least one lead or carrying at least a partial number of the electrodes.

The CNS stimulation module or the PNS stimulation module may comprise at least one electrode. It may be generally possible that the CNS stimulation module or the PNS stimulation module may comprise a plurality of electrodes. Also, it may be possible that the CNS stimulation module or the PNS stimulation module each comprise a pulse generator, e.g. an implantable pulse generator. Also, it may be possible that the stimulation unit comprises the CNS stimulation module or the PNS stimulation module. In one embodiment, the CNS stimulation module or the PNS stimulation module may be comprised in the sensing unit.

It is generally possible that the CNS stimulation module comprises an epidural stimulation module capable to provide epidural stimulation, a trans-cutaneous stimulation module capable to provide transcutaneous stimulation, a subdural stimulation module capable to provide subdural stimulation, an intracortical stimulation module capable to provide intracortical stimulation, or an intraspinal stimulation module capable to provide intraspinal stimulation. Depending on the physiological constitution of the patient and the injury or disorder of the patient, most suitable CNS stimulation, and thus the most suitable CNS stimulation module may be provided. The epidural stimulation module may comprise at least one electrode, which can be invasive and implanted in the spinal channel (epidurally). Similarly, the subdural stimulation module may comprise at least one electrode, which can be invasive and implanted subdurally. The epidural electrode(s) or subdural electrode(s) can be implanted in the vertebral channel through minimally invasive or invasive surgical techniques.

However, the system is not limited to such an application. An intracortical stimulation module may comprise at least one electrode and can be invasive and implanted intracortically. A transcutaneous stimulation module can comprise at least one electrode, which can be non-invasive and may be placed on the neck or back of the patient treated with the system, along the spine. Generally speaking, stimulation electrode arrays can also be non-invasive, e.g. by administering and providing transcutaneous stimulation to the spinal cord or other parts of the nervous system.

The PNS stimulation module may comprise at least one electrode, which can be invasive and implanted, implantable, or noninvasive, wherein noninvasive means that stimulation is provided transcutaneously. PNS stimulation may be applied to the limbs or the abdomen of a patient. The electrodes may be, for instance, placed on the abdomen or limbs (legs, arms) of a patient treated with the system. In some embodiments, the electrodes, implanted or non-implanted, may be placed closely to large vessels of the patient.

Consistent with disclosed embodiments, the PNS stimulation module may be a Functional Electrical Stimulation (FES) module capable to provide electrical stimulation to peripheral nerves or muscles.

In some embodiments, PNS stimulation may be applied to directly evoke vasoconstriction or vasodilatation by contraction/dilatation of smooth muscles of the vessels, or to evoke blood pressure alterations by stimulation of skeletal muscles.

In some embodiments, the CNS stimulation module may be at least partially implantable or at least partially implanted, or that the PNS stimulation module may be at least partially implantable or at least partially implanted. This means that at least one of the electrodes, the electrode array, the pulse generator of the CNS stimulation module, or the PNS stimulation module may be implantable. The implantable pulse generator(s) or electrodes (e.g. in the form of an electrode paddle with an array of electrodes) can be implanted minimally invasive or invasive. This has the advantage of improved ability of the patient treated with the system to function and participate in activities of daily living.

In some embodiments, the components of the system for neuromodulation or neurostimulation may form a closed-loop system. This has the advantage that the stimulation provided by the system may be adapted to specific needs or condition of the patient, the stimulation being adjusted according to feedback data of the patient, provided via the sensing unit.

In some alternative embodiments, the components of the system for neuromodulation or neurostimulation may form an open-loop system. Consistent with disclosed embodiments, open-loop may be understood as delivery of pre-programmed spatiotemporal stimulation or pre-programmed spatiotemporal stimulation patterns with spatial and temporal components. With such an open-loop system open-loop phasic stimulation may be provided. In contrast to closed-loop systems, open-loop may be understood such that neuromodulation or neurostimulation is provided, but the feedback from the patient is not used or does not influence the stimulation data. Also, the stimulation provided by the stimulation unit, inter alia the sequences provided, is/are maintained. This allows a simplified and reliable system. Also, the system may be less complex. It can form an additional system or supplement for existing systems or other systems.

In some embodiments, the components of the system for neuromodulation or neurostimulation form partially an open-loop system and partially closed-loop system. For instance in a non-limiting way, the system may be configured such that the stimulation data may be re-configured or adjusted based on data being delivered by the closed-loop system, especially wherein the re-configuration or adjustment is done in real-time. In some embodiments, the closed-loop system and its data may be used to adapt the system to be an open-loop system. Such closed-loop system data may be delivered to the control unit, which then may modify the stimulation data. In other words, the closed-loop system may be used to re-configure or adjust the stimulation data of the open-loop system.

In some embodiments, the control unit is configured such that control may be done in real-time. Real-time may be understood as real-time or close to real-time. For instance, in a non-limiting way, a time frame and short delay between 0.0 to e.g. approximately 50 ms can be understood to fulfill the condition real-time. However, shorter or longer time delays can also be understood to fulfill the condition real-time. In some embodiments, the closed-loop system may work in real-time or may be a real-time system such that feedback data being sensed by the system (more precisely the sensing unit) are processed as input variable for control of the system and that this processing is done in real-time. Overall, this may enable that the stimulation of the patient is adapted immediately to the specific needs and status of the patient. Thus, consequences of hypotension or hypertension, such as loss of conciseness, dizziness, vascular damage, occurring because the stimulation is not immediately (in real-time) adapted to the patient's blood pressure status may be avoided. In some embodiments, this may be advantageous when the patient is in motion, for example in a non-limiting way, changing from lying to sitting or standing.

In some embodiments, the system may comprise a CNS stimulation module and a PNS stimulation module. This combination of CNS and PNS stimulation may allow best possible stimulation, which overcomes the drawbacks of providing CNS stimulation or PNS stimulation only.

Consistent with disclosed embodiments, the control unit may be configured such that the PNS stimulation provided by the PNS stimulation module and the CNS stimulation provided by the CNS stimulation module is at least partially interleaved. With this, at least two programming settings may be interleaved, and specific patient requirements may be reflected, side effects may be avoided. By a non-limiting example, this feature may allow for shaping of the individualized current fields provided by the CNS stimulation module to fall below the side effect threshold and prevent stimulation of nontargeted anatomical regions and its adjacent structures, thereby reducing side effects and preserving stimulation benefits, and the CNS stimulation provided by the CNS stimulation may be interleaved with PNS stimulation provided to peripheral target zones of the patient, where higher stimulation may be required.

Consistent with disclosed embodiments, the control unit may be configured such that the PNS stimulation provided by the PNS stimulation module and the CNS stimulation provided by the CNS stimulation module is at least partially superimposed. In some embodiments, this may enable that side effects may be avoided or compensated. As a non-limiting example, this feature may allow for shaping of the individualized current fields provided by the PNS stimulation module to compensate for side effects of nontargeted anatomical regions or its adjacent structures caused by stimulation provided by the CNS stimulation module. Overall, this may enable preserving stimulation benefits.

Consistent with disclosed embodiments, the control unit may be capable to independently control and switch on and off either the PNS stimulation module or the CNS stimulation module. It may be possible that switch on/off mechanism is based on physiological data of the patient provide by the sensing unit, or feedback information.

Alternatively, or additionally, a stimulation program may comprise time-regulated PNS or CNS stimulation. Overall, this may allow most flexible stimulation of a patient, and further contribute to limit side effects provided the stimulation. Further, this may extend the duration of the energy supply of the implanted pulse generator via a battery, thus reducing costs and effort.

Consistent with disclosed embodiments, a first system for neuromodulation or neurostimulation may be combined with at least one further system for neuromodulation or neurostimulation for providing neurostimulation to the patient (e.g. a system providing neurostimulation for restoring motor function of the patient), wherein the control unit of the first system is configured and arranged to trigger that the PNS stimulation provided by the PNS stimulation module or the CNS stimulation provided by the CNS stimulation module of the first system is at least partially used for correction of effects of stimulation provided by the at least one further system, to, as a non-limiting example, refine motor output. In some embodiments, this may enable that side effects caused by stimulation provided to restore motor function be avoided or compensated. In other words, a patient suffering from motor dysfunction and autonomic dysfunction caused by SCI may be stimulated with a stimulation module in order to restore motor function. This stimulation may cause side effects on autonomic function, including but not limited to sudden blood pressure elevations/decreases, sudden effect on respiration, uncontrolled bowel or bladder sphincter relaxation, uncontrolled sexual function. These side effects may be at least partially avoided, limited, or compensated by stimulation provided with the CNS stimulation module or the PNS stimulation module.

The stimulation provided with the CNS stimulation module or the PNS stimulation module may be superimposed to the stimulation provided with other stimulation module to refine motor output. Otherwise, the stimulation provided with the CNS stimulation module or the PNS stimulation module and the stimulation provided with other stimulation module to refine motor output may be interleaved.

The system may further comprise at least one of a processor, a telemetry module, a feedback module, or a further system for providing neuromodulation.

According to the present disclosure, a method is disclosed for providing neuromodulation or neurostimulation by providing CNS stimulation combined with PNS stimulation, by using a system for neuromodulation or neurostimulation.

Further, the present disclosure relates to the use of a system for the treatment of a patient suffering from neurological dysfunction, including autonomic dysfunction, after spinal cord injury, traumatic brain injury, cerebral palsy, stroke, Parkinson Disease, multiple sclerosis, autonomic failure, autonomic neuropathy, or cancer of the neurological tissue.

Further details and advantages of the present disclosure shall now be disclosed in connection with the figures.

### FIGURES

FIG. 1 a schematic overview of an embodiment of a system for neuromodulation or neurostimulation according to the present disclosure, with which the method according to the present disclosure can be performed; and

FIG. 2 a schematic overview of a further embodiment of the system according to the present disclosure, with which the method according to the present disclosure can be performed.

### BRIEF DESCRIPTIONS OF THE DRAWINGS

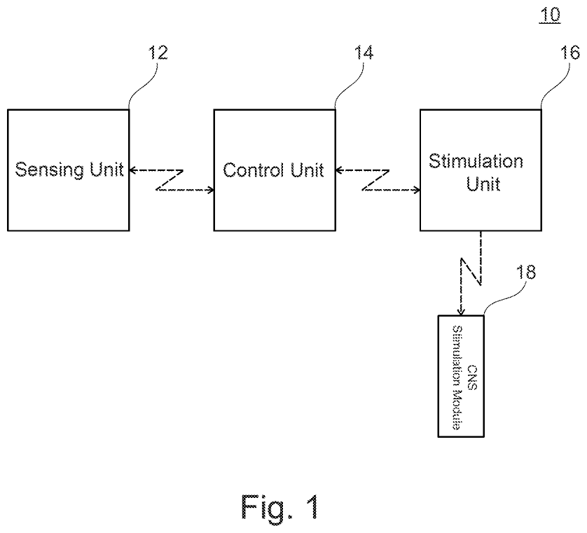

FIG. 1 shows a schematic overview of an embodiment of the system for neuromodulation or neurostimulation 10 according to the present disclosure, with which the method according to the present disclosure can be performed.

In this embodiment, a system for neuromodulation or neurostimulation 10 is shown. The system 10 comprises a sensing unit 12. In this embodiment, the sensing unit 12 comprises a blood pressure sensor. In this embodiment, the sensing unit 12 comprises a finger blood pressure monitoring system. However, any other type of blood pressure sensor could be generally possible.

In an alternative embodiment, the system 10 can comprise more than one sensing unit 12. The sensing unit 12 could additionally or alternatively comprise a pulse oximeter, a heart-rate sensor, a breathing rate sensor, a body temperature sensor, a pressure sensor (for instance for sensing bladder internal pressure), a pH sensor, a barometer, an inertial measurement unit, an accelerometer, a gyroscope, a chemical concentration sensor (glucose, water, sodium, calcium), a hormone sensor, a metabolites sensor, a sweat sensor, a gastrointestinal movement sensor, a hydration sensor, an electrolyte sensor, an arterial stiffness sensor, a urine concentration sensor, a urine volume sensor, a bladder volume sensor, a conductivity sensor, a capacitance sensor, a strain gauge sensor, or a genital blood flow sensor.

In this embodiment, the system 10 further comprises a control unit 14. The system 10 may further comprise a stimulation unit 16. The stimulation unit 16 may be an implantable pulse generator. It is generally possible that, alternatively or additionally, other components of the system 10 are implantable. The system 10 may further comprise a CNS stimulation module 18. The CNS stimulation module 18 may comprise an electrode array for providing CNS stimulation, EES, or both. In other words, the CNS stimulation module may comprise an epidural stimulation module capable to provide epidural stimulation, in some embodiments, epidural electrical stimulation.

Consistent with disclosed embodiments, the electrode array may comprise a plurality of electrodes. In some embodiments, the electrode array comprises 16 electrodes. However, the electrode array can generally comprise n electrodes, with n being any natural number >0.

The sensing unit 12 may be connected to the control unit 14. This connection is a direct connection. In some embodiments, this connection can be a bidirectional connection. In some embodiments, this connection can be a wireless connection. The sensing unit 12 may provide a sensor signal correlating with blood pressure of a patient equipped with the system 10. The sensing unit 12 may provide a sensor signal indicating systolic blood pressure of the patient equipped with the system 10.

In general, the sensing unit 12 can provide a sensor signal correlating with a physiological value, which describes neurological function or dysfunction of a patient. In some embodiments, the physiological value describes autonomic function or dysfunction of a patient.

The sensor signal may be provided to the control unit 14. The control unit 14 detects hypotension of the patient based on the sensor signal. The control unit 14 can trigger neurostimulation for stabilization of blood pressure of the patient.

The control unit 14 is connected to the stimulation unit 16. This connection is a direct connection. Further, this connection is also a bidirectional connection and a wireless connection.

In general, the control unit 14 can detect a dysfunction of neurological function, preferably autonomic function of the patient based on the sensor signal and to trigger neuromodulation for stabilization or treatment of the neurological function, preferably autonomic function of the patient.

The stimulation unit 16 is connected to the CNS stimulation module 18. This connection is a direct connection. Further, this connection is also a unidirectional connection and a cable-bound connection.

In an alternative embodiment, the system 10 can comprise more than one control unit 14. Similarly, the system 10 can comprise more than one stimulation unit 16. In some embodiments, the stimulation unit 16 could be a non-implantable pulse-generator.

In other alternative embodiments, the system 10 could additionally or alternatively comprise a PNS stimulation module 20. The PNS stimulation module 20 could be a Functional Electrical Stimulation (FES) module capable to provide electrical stimulation of peripheral nerves.

In other alternative embodiments, the connection between the sensing unit 12 and the control unit 14 could be an indirect connection. The connection could be a unidirectional connection. The connection could be a cable-bound connection.

In other alternative embodiments, the connection between the control unit 14 and the stimulation unit 16 could be an indirect connection. The connection could be a unidirectional connection. The connection could be a cable-bound connection.

In other alternative embodiments, the connection between the stimulation unit 16 and the CNS stimulation module 18 could be an indirect connection. The connection could be a unidirectional connection. The connection could be a cable-bound connection.

In an alternative embodiment, the components of the system 10 can form an open-loop system 10. In another alternative system 10, the components of the system 10 can form partially an open-loop system and partially a closed-loop system.

Not shown in FIG. 1 is that alternatively, or additionally, to the epidural stimulation module, the CNS stimulation module 18 could comprise a transcutaneous stimulation module capable to provide transcutaneous stimulation, a subdural stimulation module capable to provide subdural stimulation, an intracortical stimulation module capable to provide intracortical stimulation, or an intraspinal stimulation module capable to provide intraspinal stimulation.

Further not shown in FIG. 1 is that the sensing unit 12 could alternatively or additionally comprise an electrocardiogram (ECG). The sensing unit 12 could also alternatively or additionally comprise an electroencephalogram (EEG), an electromyogram (EMG), a patch clamp, a voltage clamp, an extracellular single-unit recorder, or a recorder of local field potentials.

Further not shown in FIG. 1 is that the sensing unit 12 could additionally or alternatively comprise at least one trigger, e.g. a manual trigger, to be triggered by the patient and providing a signal describing dysfunction of the autonomic function, e.g. blood pressure, of the patient.

Further not shown in FIG. 1 is that the sensing unit 12 or the control unit 14 can optimize stimulation parameters for stabilization or treatment of the neurological function of the patient. In some embodiments, the neurological function may be an autonomic function of the patient.

In general, the system 10 can be used in a method for the treatment of a patient suffering from neurological dysfunction, including autonomic dysfunction, after spinal cord injury, traumatic brain injury, cerebral palsy, stroke, Parkinson Disease, multiple sclerosis, autonomic failure, autonomic neuropathy, cancer of the neurological tissue, or any other disease impairing the nervous system of the patient.

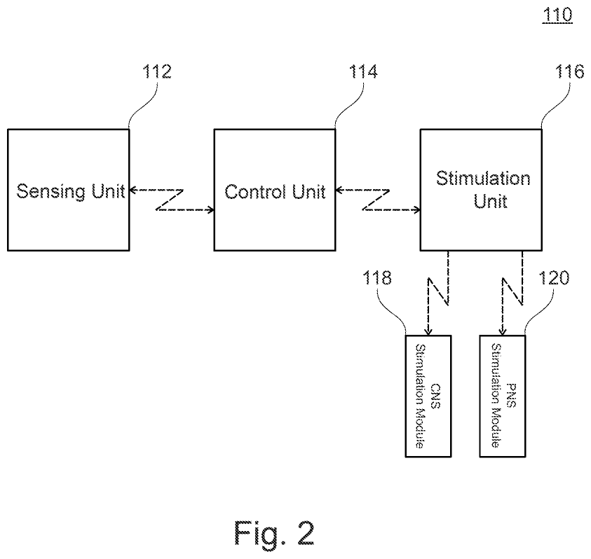

FIG. 2 shows a schematic overview of a further embodiment of the system 110 according to the present disclosure, with which the method according to the present disclosure can be performed.

The system 110 comprises the structural and functional features of the system 10 disclosed in FIG. 1. The corresponding reference numbers in FIG. 2 for identical or similar elements or features correspond to the corresponding reference numbers of FIG. 1 and to reflect this, then these reference numbers are taken and increased by the value 100.

In addition to the CNS stimulation module 118 the system 110 further comprises a PNS stimulation module 120. The stimulation unit 116 is connected to the CNS stimulation module 118 and the PNS stimulation module 120. Both connections are direct connections. Further, both connections are unidirectional connections. However, alternatively, the connection between the stimulation unit 116 and the CNS stimulation module 118 or the stimulation unit 116 or the PNS stimulation module 120 could be an indirect or bidirectional connection.

The connection between the stimulation unit 116 and the CNS stimulation module 118 is a cable-bound connection. However, in an alternative embodiment, this connection could be a wireless connection.

The connection between the stimulation unit 116 and the PNS stimulation module 120 is a wireless connection. However, in an alternative embodiment, this connection could be a cable-bound connection.

The CNS stimulation module 118 and the PNS stimulation module 120 are indirectly connected (via the stimulation unit 116). Not shown in FIG. 2 is that the CNS stimulation module 118 and the PNS stimulation module 120 could be directly connected (wireless or cable-bound connection, unidirectional or bidirectional connection).

The CNS stimulation module 118 is an epidural stimulation module for providing EES. The CNS stimulation module 118 comprises an electrode array, which is implantable.

The PNS stimulation module 120 is a Functional Electrical Stimulation (FES) module capable to provide electrical stimulation to peripheral nerves. In an alternative embodiment, the PNS stimulation module 120 provide electrical stimulation to smooth muscles of blood vessels.

The FES module provides stimulation transcutaneously. The FES module comprises an electrode placed transcutaneously and providing electrical stimulation to the lower legs of a patient. Alternatively, the FES module could be implantable or have the electrodes implanted. In general, the FES module could comprise a plurality of electrodes. The CNS stimulation module 118 can be at least partially implantable or at least partially implanted. The PNS stimulation module 120 can be at least partially implantable or at least partially implanted.

In this embodiment, the control unit 114 controls the stimulation provided by the PNS stimulation module 120 and the CNS stimulation module 118 such that the stimulation provided by the two stimulation modules 118, 120 is interleaved. In other words, the control unit 114 is configured such that the PNS stimulation provided by the PNS stimulation module 120 and the CNS stimulation provided by the CNS stimulation module 118 are least partially interleaved. Alternatively, the control unit 114 can be configured such that the PNS stimulation provided by the PNS stimulation module 120 and the CNS stimulation provided by the CNS stimulation module 118 is at least partially superimposed. In general, the system 110 can be used for a method for providing neuromodulation or neurostimulation by providing CNS stimulation combined with PNS stimulation.

Not shown in FIG. 2 is that it is generally possible that the control unit 114 can independently control and switch on and off either the PNS stimulation module 120 or the CNS stimulation module 118. Further not shown in FIG. 2 is that the system 110 can be a first system 110 which is configured and arranged to be combined with at least one further system for providing neurostimulation to the patient, wherein the control unit 114 of the first system 110 is configured and arranged to trigger that the PNS stimulation provided by the PNS stimulation module 120, or the CNS stimulation provided by the CNS stimulation module 118 of the first system 110, is at least partially used for correction of effects of stimulation provided by the at least one further system.

The foregoing descriptions have been presented for purposes of illustration. They are not exhaustive and are not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

It should be noted that, the relational terms herein such as “first” and “second” are used only to differentiate an entity or operation from another entity or operation, and do not require or imply any actual relationship or sequence between these entities or operations. Moreover, the words “comprising,” “having,” “containing,” and “including,” and other similar forms are intended to be equivalent in meaning and be open ended in that an item or items following any one of these words is not meant to be an exhaustive listing of such item or items or meant to be limited to only the listed item or items.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

As used herein, unless specifically stated otherwise, the terms “and/or” and “or” encompass all possible combinations, except where infeasible. For example, if it is stated that a database may include A or B, then, unless specifically stated otherwise or infeasible, the database may include A, or B, or A and B. As a second example, if it is stated that a database may include A, B, or C, then, unless specifically stated otherwise or infeasible, the database may include A, or B, or C, or A and B, or A and C, or B and C, or A and B and C.

It is appreciated that the above-described embodiments can be implemented by hardware, or software (program codes), or a combination of hardware and software. If implemented by software, it may be stored in the above-described computer-readable media. The software, when executed by the processor can perform the disclosed methods. The computing units and other functional units described in this disclosure can be implemented by hardware, or software, or a combination of hardware and software. One of ordinary skill in the art will also understand that multiple ones of the above-described modules/units may be combined as one module/unit, and each of the above-described modules/units may be further divided into a plurality of sub-modules/sub-units.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the disclosure disclosed herein. It is intended that the specification and examples be considered as exemplary only, with a true scope and spirit of the disclosure being indicated by the following claims. It is also intended that the sequence of steps shown in figures are only for illustrative purposes and are not intended to be limited to any particular sequence of steps. As such, those skilled in the art can appreciate that these steps can be performed in a different order while implementing the same method.

What is claimed is:

1. A system for neuromodulation or neurostimulation comprising:
at least one sensing unit configured to provide a sensor signal correlating with a physiological value describing neurological function or dysfunction of a patient;
at least one control unit;
at least one stimulation unit; and
at least one of
at least one Central Nervous System stimulation module for providing Central Nervous System stimulation; or
at least one Peripheral Nervous System stimulation module for providing Peripheral Nervous System stimulation;
wherein the control unit is configured to detect the neurological dysfunction based on the sensor signal and to trigger the neuromodulation or neurostimulation.

2. The system of claim 1, wherein at least one of the components of the system is implantable.

3. The system of claim 1, wherein the at least one sensing unit comprises:
at least one of an electrocardiogram, an electroencephalogram, or an electromyogram;
a patch clamp;
a voltage clamp;
an extracellular single-unit recorder; or
a recorder of local field potentials.

4. The system of claim 1, wherein the at least one sensing unit comprises at least one of a blood pressure sensor, a pulse oximeter, a heart-rate sensor, a breathing rate sensor, a body temperature sensor, a pressure sensor, a pH sensor, a barometer, an inertial measurement unit, an accelerometer, a gyroscope, a chemical concentration sensor (glucose, water, sodium, calcium), a hormone sensor, a metabolites sensor, a sweat sensor, a gastrointestinal movement sensor, a hydration sensor, an electrolyte sensor, an arterial stiffness sensor, a urine concentration sensor, a urine volume sensor, a bladder volume sensor, a conductivity sensor, a capacitance sensor, a strain gauge sensor, or a genital blood flow sensor.

5. The system of claim 1, wherein the at least one of the at least one sensing unit and the control unit is configured to optimize stimulation parameters for stabilization, or treatment, or both, of the neurological function of a patient.

6. The system of claim 1, wherein the at least one sensing unit comprises at least one trigger configured to be triggered by a patient and providing a signal describing dysfunction of an autonomic function of a patient.

7. The system of claim 6, wherein the at least one trigger is a manual trigger.

8. The system of claim 1, wherein the Central Nervous System stimulation module comprises at least one of:
an epidural stimulation module capable to provide epidural stimulation;
a trans-cutaneous stimulation module capable to provide trans-cutaneous stimulation;
a subdural stimulation module capable to provide subdural stimulation;
an intracortical stimulation module capable to provide intracortical stimulation; or
an intraspinal stimulation module capable to provide intraspinal stimulation.

9. The system of claim 1, wherein the Peripheral Nervous System stimulation module is a Functional Electrical Stimulation module capable to provide electrical stimulation of peripheral nerves.

10. The system of claim 1, wherein
the Central Nervous System stimulation module is at least partially implantable or at least partially implanted; or
the Peripheral Nervous System stimulation module is at least partially implantable or at least partially implanted; or
the Central Nervous System stimulation module is at least partially implantable or at least partially implanted, and the Peripheral Nervous System stimulation module is at least partially implantable or at least partially implanted.

11. The system of claim 1, wherein the system is a closed-loop system.

12. The system of claim 1, wherein the system is an open-loop system.

13. The system of claim 1, wherein the components of the system form partially an open-loop system and partially closed-loop system.

14. The system of claim 1, wherein the control unit is configured for real-time control.

15. The system of claim 1, wherein the control unit is configured such that the Peripheral Nervous System stimulation provided by the Peripheral Nervous System stimulation module and the Central Nervous System stimulation provided by the Central Nervous System stimulation module is at least partially interleaved.

16. The system of claim 1, wherein the control unit is configured such that the Peripheral Nervous System stimulation provided by the Peripheral Nervous System stimulation module and the Central Nervous System stimulation provided by the Central Nervous System stimulation module is at least partially superimposed.

17. The system of claim 1, wherein the control unit is capable to independently control and switch on and off either the Peripheral Nervous System stimulation module or the Central Nervous System stimulation module.

18. The system of claim 1, wherein the system is configured to be combined with at least one additional system of claim 1,
wherein the control unit is configured to trigger that at least one of the Peripheral Nervous System stimulation provided by the Peripheral Nervous System stimulation module or the Central Nervous System stimulation provided by the Central Nervous System stimulation module of the system is at least partially used for correction of effects of stimulation provided by the at least one additional system of claim 1.

19. A method for providing at least one of neuromodulation or neurostimulation to treat a patient's neurological dysfunction by using a system, the system for neuromodulation or neurostimulation comprising:
at least one sensing unit configured to provide a sensor signal correlating with a physiological value describing the neurological dysfunction;
at least one control unit;
at least one stimulation unit; and
at least one of
at least one Central Nervous System stimulation module for providing Central Nervous System stimulation; or
at least one Peripheral Nervous System stimulation module for providing Peripheral Nervous System stimulation;
wherein the control unit is configured to detect the neurological dysfunction on the sensor signal and to trigger at least one of neuromodulation for stabilization or treatment of the neurological function of the patient.

20. The method of claim 19, the patient's neurological dysfunction comprises an autonomic dysfunction, an after-spinal cord injury, a traumatic brain injury, cerebral palsy, stroke, Parkinson Disease, multiple sclerosis, autonomic failure, autonomic neuropathy, or cancer of the neurological tissue.
